(19) United States
(12) Patent Application Publication
(10) Pub. No.: US 2025/0215005 A1
(43) Pub. Date: Jul. 3, 2025

(54) PROCESS FOR PREPARATION OF RUXOLITINIB

(71) Applicant: Glenmark Life Sciences Limited, Solapur (IN)

(72) Inventors: Venkata Raghavendra Acharyulu PALLE, Pune (IN); Subbiah RAMAR, Vadodara (IN); Vishweshwar PEDDY, Hyderabad (IN); Suresh Babu NARAYANAN, Dombivli (IN); Premchand PATIL, Dhule (IN); Amit Anant THANEDAR, Dombivali (IN); Rahul Bhalchandra KAWTHEKAR, Dombivali (IN); Rajendra Singh SHEKHAWAT, Dombivali (IN); Santosh CRASTA, Mulund (IN)

(21) Appl. No.: 18/867,160

(22) PCT Filed: May 18, 2023
(86) PCT No.: PCT/IB2023/055117

§ 371 Date: Nov. 19, 2024

(30) Foreign Application Priority Data
May 19, 2022 (IN) 202221028894

(51) Int. Cl. C07D 487/04

(52) U.S. Cl. CPC C07D 487/04

(57) ABSTRACT

The present invention provides a process for the preparation of ruxolitinib or a pharmaceutically acceptable salt thereof. In particular, the present invention provides a process for the preparation of crystalline (R)-ruxolitinib phosphate. The present invention also provides pharmaceutical composition comprising crystalline (R)-ruxolitinib phosphate which is obtained by the process of the present invention. The present invention provides a compound of formula IV.

### PRIORITY

This application claims the benefit of Indian Provisional Application No. 202221028894 filed on May 19, 2022, entitled “Process for preparation of ruxolitinib”, the contents of which are incorporated herein by reference.

### FIELD OF INVENTION

The present invention provides a process for the preparation of ruxolitinib or a pharmaceutically acceptable salt thereof. In particular, the present invention provides a process for the preparation of crystalline (R)-ruxolitinib phosphate. The present invention also provides pharmaceutical composition comprising the crystalline (R)-ruxolitinib phosphate, which is obtained by the process of the present invention.

### BACKGROUND OF THE INVENTION

Ruxolitinib is known by its chemical name (R)-3-(4-(7H-pyrrolo [2,3d]pyrimidin-4-yl)-1H-pyrazol-1-yl)-3-cyclopentylpropanenitrile, and is represented by a compound of formula I (the “compound I”),

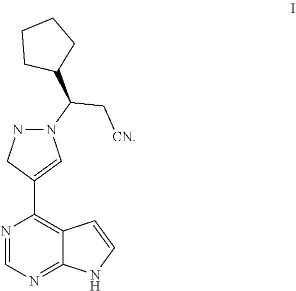 [SMILES: N#CC[C@H](C1CCCC1)N1C=C(c2ncnc3[nH]ccc23)CN1]

Ruxolitinib is a Janus kinase inhibitor (JAK Inhibitor), and marketed as ruxolitinib phosphate under the brand name Jakafi® by Incyte Corp.

Ruxolitinib and its pharmaceutically acceptable salts including the phosphate salt, are described in a published PCT application no. WO 2007070514.

Ruxolitinib phosphate is indicated for the treatment of patients with intermediate or high-risk myelofibrosis, including primary myelofibrosis, post-polycythemia vera myelofibrosis and post-essential thromocythemia myelofibrosis.

Various processes for the synthesis of ruxolitinib and its pharmaceutically acceptable salts including the phosphate salt are known in the art. For instance, the PCT Publication No WO2010083283, and the U.S. patent Ser. No. 10/562,904 disclose processes for the synthesis of ruxolitinib.

### SUMMARY OF THE INVENTION

The present invention provides a process for the preparation of crystalline (R)-3-(4-(7H-pyrrolo[2,3-d] pyrimidin-4-yl)-1H-pyrazol-1-yl)-3-cyclopentylpropanenitrile phosphate, a compound of formula I-A (the “compound I-A” or “(R)-ruxolitinib phosphate”),

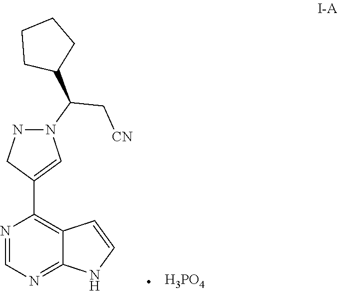

comprising the steps of:
a) reacting 3-cyclopentyl-3-{4-[7-(triphenylmethyl)-pyrrolo[2,3-d]pyrimidin-4-yl]-1H-pyrazol-1-yl} propanenitrile a compound of formula IV (the “compound IV”);

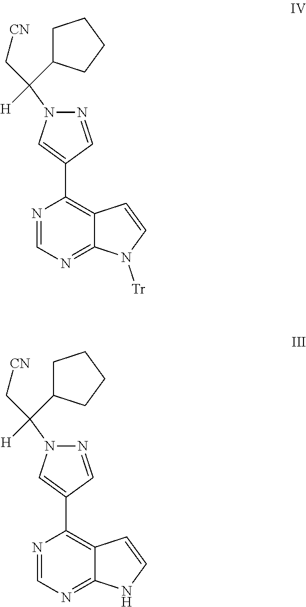

with an acid under anhydrous condition to obtain racemic ruxolitinib, a compound of formula III (the “compound III”);
b) reacting the compound III obtained in the step (a) with a chiral acid to obtain the chiral salt of ruxolitinib with the chiral acid, represented by the compound of formula II (the “compound II”),

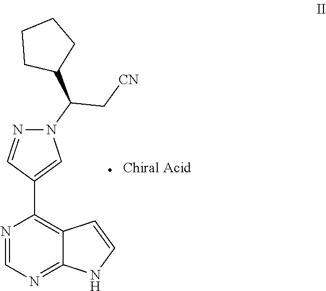 [SMILES: N#CC[C@H](C1CCCC1)n1cc(-c2ncnc3[nH]ccc23)cn1 ; "Chiral Acid"]

wherein the chiral acid is as described herein; and
c) converting the resulting chiral salt of ruxolitinib (the compound II) to crystalline (R)-ruxolitinib phosphate.

The present invention provides a compound of formula IV (the “compound IV”),

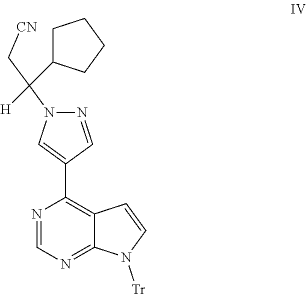

wherein “Tr” is triphenylmethyl.

The present invention also provides two impurities, i.e., compounds of formulae X (the “compound X”) and XI (the “compound XI”) having the following chemical structures;

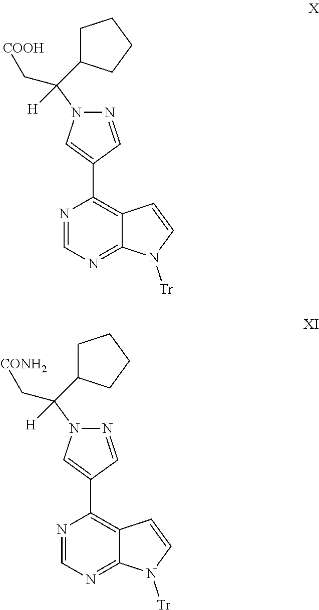

wherein “Tr” is triphenylmethyl.

The present invention also provides pharmaceutical composition comprising the crystalline (R)-ruxolitinib phosphate, which is as described herein.

### BRIEF DESCRIPTION OF DRAWINGS

FIG. 1 is characteristic XRPD (X-Ray Powder Diffraction) pattern of R-ruxolitinib phosphate as obtained in example 5.

FIG. 2 is characteristic DSC (Differential Scanning Calorimetry) thermogram of R-ruxolitinib phosphate as obtained in example 5.

FIG. 3 is characteristic TGA (Thermogravimetric Analysis) thermogram of R-ruxolitinib phosphate as obtained in example 5.

FIG. 4 is characteristic XRPD (X-Ray Powder Diffraction Pattern) of R-ruxolitinib DBTA ((+)-dibenzoyl-D-tartaric acid) salt as obtained in example 4B.

FIG. 5 is characteristic DSC (Differential Scanning Calorimetry) thermogram of R-ruxolitinib DBTA salt as obtained in example 4B.

FIG. 6 is characteristic TGA (Thermogravimetric Analysis) thermogram of R-ruxolitinib DBTA salt as obtained in example 4B.

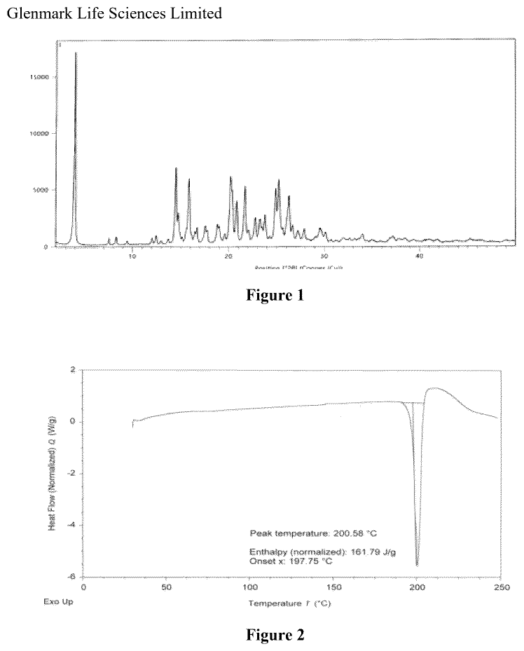

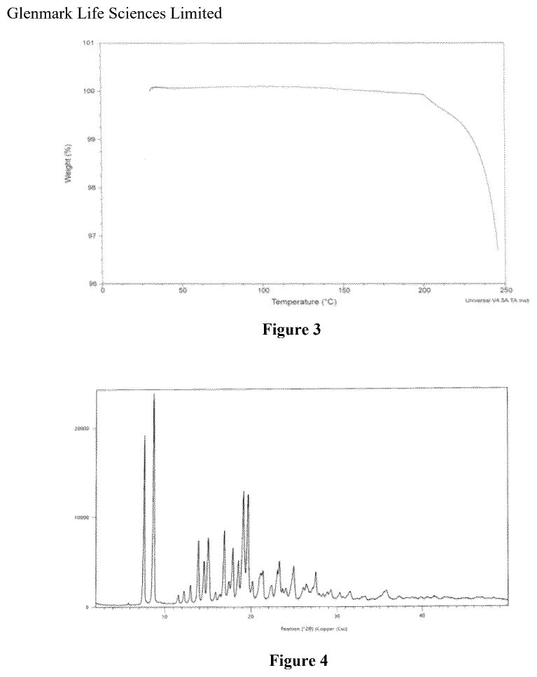

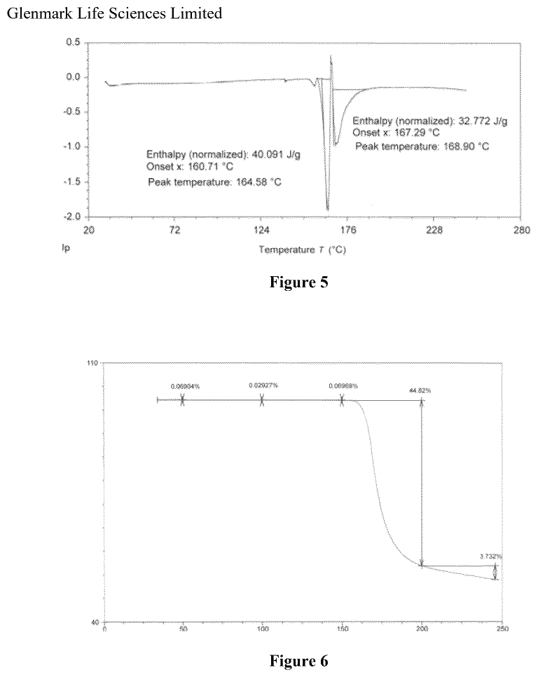

### DETAILED DESCRIPTION OF THE INVENTION

In one aspect, the present invention relates to a process for the preparation of crystalline (R)-3-(4-(7H-pyrrolo[2,3-d]pyrimidin-4-yl)-1H-pyrazol-1-yl)-3-cyclopentylpropanenitrile phosphate, a compound of formula I-A (the “compound I-A” or “(R)-ruxolitinib phosphate”),

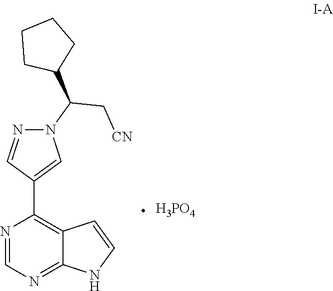

comprising the steps of:
a) reacting 3-cyclopentyl-3-{4-[7-(triphenylmethyl)-pyrrolo[2,3-d]pyrimidin-4-yl]-1H-pyrazol-1-yl}propanenitrile, a compound of formula IV (the “compound IV”) with an acid under anhydrous condition to obtain racemic ruxolitinib, a compound of formula III (the “compound III”);

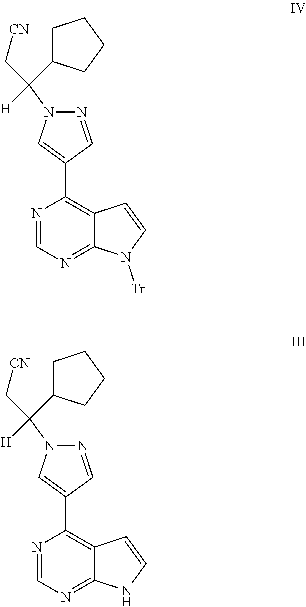

wherein “Tr” is triphenylmethyl;
b) reacting the compound III obtained in the step (a) with a chiral acid to obtain a chiral salt of ruxolitinib with the chiral acid represented by a compound of formula II (the “compound II”); and

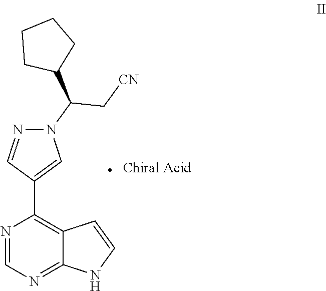 [SMILES: N#CC[C@H](C1CCCC1)n1cc(-c2ncnc3[nH]ccc23)cn1 ; "Chiral Acid"]

c) converting the resulting chiral salt of ruxolitinib, the compound II obtained in the step (b) to crystalline (R)-ruxolitinib phosphate, the compound I-A.

The term “anhydrous condition” as used herein refers to an environment wherein the moisture content is less than 0.2% as measured by the known Karl Fischer method. The anhydrous condition is such that compound of formula IV will not get converted to the two impurities, i.e., compounds of formulae X (the “compound X”) or XI (the “compound XI”)

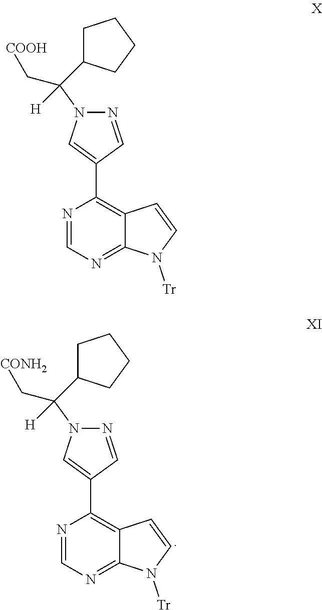

The anhydrous condition is achieved by addition of a dehydrating agent to the reaction mixture of the step (a).

In one embodiment, the dehydrating agent is selected from the group consisting molecular sieves, sodium sulfate, magnesium sulfate and the like.

Alternatively, the anhydrous condition can be achieved by carrying out the reaction in the step (a) in the presence of an agent that inhibits the formation of the compound X or the compound XI.

In an embodiment, the agent that inhibits the formation of the compound X and/or the compound XI is an alcoholic solvent.

In an embodiment, the alcoholic solvent may be selected from the group consisting of methanol, ethanol, ethane-1,2-diol, n-propanol, isopropanol, n-butanol, and the like.

In one embodiment, in the step a) of the above process, the acid may be selected from the group consisting of formic acid, acetic acid, citric acid, tartaric acid, bi-tartaric acid, benzoic acid, lactic acid, oxalic acid, malic acid, fumaric acid, succinic acid, gluconic acid, pamoic acid, methanesulfonic acid, benzenesulfonic acid, triflic acid, haloacetic acid, anhydrous HCl, sulphuric acid, hydrobromic acid in acetic acid and the like.

In an embodiment, the compound III obtained in the step (a) may be optionally purified using acid-base purification method.

The compound III obtained in the step (a) is racemic mixture of ruxolitinib base, which may also be referred to herein as racemate of ruxolitinib or racemic ruxolitinib.

In one embodiment, the step (a) may be carried out in the presence of a solvent.

In one embodiment, the solvent used in the step (a) includes, but is not limited to, nitriles such as acetonitrile, propionitrile; hydrocarbons such as toluene, xylene, heptane, hexane, cyclohexane and the like; esters such as methyl acetate, ethyl acetate, n-propyl acetate, isopropyl acetate, butyl acetate, tert-butyl acetate and the like; ethers such as diethyl ether, diisopropyl ether, methyl tert-butyl ether, tetrahydrofuran and the like; chlorinated solvents such as methylene dichloride, ethylene dichloride and the like; ketones such as acetone, ethyl methyl ketone, methyl isobutyl ketone; alcohols such as methanol, ethanol, n-propanol, isopropanol, n-butanol and the like.

In one embodiment, the step (a) may be carried out at a temperature ranging from about 25° C. to about reflux temperature of the solvent used.

In one embodiment, in the step (b) of the above process, the compound III is reacted with a chiral acid to obtain the corresponding chiral salt of ruxolitinib with the chiral acid, the compound II, in the presence of a solvent.

In one embodiment, in the step (b) of the above process the compound III remains insitu i.e. it is carried over to the next step without isolation.

Alternatively, in the step (b) of the above process, the compound III may be isolated.

The chiral acid used in the step (b) may be selected from the group consisting of (+)-dibenzoyl-D-tartaric acid, (−)-dibenzoyl-L-tartaric acid, (+)-di-p-toluoyl-D-tartaric acid, (−)-di-p-toluoyl-L-tartaric acid, S-(+) mandelic acid, R-(−) mandelic acid, L-(+) tartaric acid, D-(−) tartaric acid, L-malic acid, D-malic acid, D-maleic acid, (R)-(−)-camphor sulfonic acid, (lS)-(+)-camphor sulfonic acid, L(−)-pyroglutamic acid, L(+)-pyroglutamic acid, and (−)-lactic acid.

In one embodiment, the chiral acid used in the step (b) is (+)-dibenzoyl-D-tartaric acid (DBTA). Accordingly, in an embodiment, (R)-ruxolitinib DBTA salt represented by the following compound of formula XIV (the “compound XIV”);

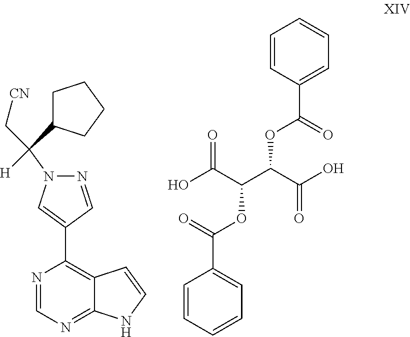

is obtained in the step (b) of the above process.

In one embodiment, the present invention provides the compound XIV which is characterized by ¹H NMR (DMSO, 400 MHz) having peaks at 13.96-13.90, 12.12, 8.81, 8.68, 8.37, 8.06-8.01, 7.76-7.71, 7.63-7.55, 6.99-6.99, 5.87, 4.57-4.51, 3.34-3.17, 2.50-2.41, 1.84-1.78, and 1.58-1.33.

In the step (b) of the process of the present invention, the compound XIV is obtained as a crystalline form, which is designated herein as “crystalline Form G1” or “Form G1”.

In one embodiment, the crystalline Form G1 of the compound XIV of the present invention is characterized by X-ray powder diffraction pattern (XRPD) having peaks at about 7.6, 8.6, 13.8, 16.8, 21.0, and 24.8±0.2 degrees 2-theta.

In one embodiment, the crystalline Form G1 of the compound XIV of the present invention is characterized by X-ray powder diffraction pattern (XRPD) as substantially illustrated in FIG. 4.

In the context of the present invention, the term “substantially illustrated” as used in reference to FIG. 4 may be understood to relate to any crystal form of (R)-ruxolitinib DBTA salt characterized with the graphical data having small variations, as are well known to the person skilled in the art, in comparison with the FIG. 4.

Further, the chiral purity of the compounds obtained by the process of the present invention can be determined using high performance liquid chromatography (HPLC) under conditions that are generally known to those skilled in the art.

In one embodiment, the reaction in the step (b) may be carried out at a temperature ranging from about 25° C. to about reflux temperature of the solvent.

In an embodiment, the reaction may be carried out at a temperature ranging from about 60° C. to about 90° C.

As used herein, the term “about” refers to any value which lies within the range defined by a number up to 10% of the value.

In one embodiment, the reaction in the step (b) is carried out in the presence of a solvent which includes, but is not limited to, nitriles such as acetonitrile, propionitrile; chlorinated solvents such as methylene dichloride, ethylene dichloride, chloroform, carbon tetrachloride; hydrocarbons such as toluene, xylene, heptane, hexane, cyclohexane and the like; esters such as methyl acetate, ethyl acetate, n-propyl acetate, isopropyl acetate, butyl acetate, tert-butyl acetate and the like; alcohols such as methanol, ethanol, isopropanol, n-propanol, butanol and the like; ethers such as diethyl ether, diisopropyl ether, methyl tert-butyl ether, tetrahydrofuran and the like; ketones such as acetone, ethyl methyl ketone, methyl isobutyl ketone and the like; water and or mixtures thereof.

The chiral salt of ruxolitinib with a chiral acid i.e. the compound II or the specific chiral salt of ruxolitinib with DBTA designated herein as the compound XIV obtained in the step (b) of the above process may be optionally purified using a solvent selected from, but not limited to, nitrile such as acetonitrile, propionitrile and the like; alcohols such as methanol, ethanol, ethane-1,2-diol, isopropanol, n-propanol, butanol and the like; ketones such as acetone, ethyl methyl ketone, methyl isobutyl ketone and the like; ethers such as diethyl ether, diisopropyl ether, methyl tert-butyl ether, tetrahydrofuran and the like; esters such as methyl acetate, ethyl acetate, n-propyl acetate, isopropyl acetate, butyl acetate, tert-butyl acetate and the like; water and or mixtures thereof.

In one embodiment, the step (c) of the above process for obtaining (R)-ruxolitinib phosphate, the compound I-A, comprises the steps of
c-I) reacting the compound XIV obtained in step (b) with a base in the presence of a solvent to obtain (R)-ruxolitinib, the compound I; and
c-II) reacting the compound I with phosphoric acid in the presence of a solvent to obtain the compound I-A.

In an embodiment, the base used in the step (c-I) may be an inorganic base selected from the group consisting of hydroxides such as sodium hydroxide, potassium hydroxide, calcium hydroxide, lithium hydroxide; carbonate such as of sodium carbonate, potassium carbonate, calcium carbonate, lithium carbonate; alkoxide such as sodium methoxide, potassium methoxide; bicarbonates such as sodium bicarbonate, potassium bicarbonate, lithium bicarbonate; ammonia and the like.

In an embodiment, the solvent used in the steps (c-I) and (c-II) may be selected from the group consisting of chlorinated solvents such as methylene dichloride, ethylene dichloride, chloroform, carbon tetrachloride; ethers such as diethyl ether, methyl tertiary butyl ether, di-isopropyl ether, tetrahydrofuran; nitriles such as acetonitrile, propionitrile; esters such as ethyl acetate, butyl acetate, isopropyl acetate; hydrocarbon such as, cyclohexane, toluene, xylene, hexane; alcohols such as methanol, ethanol, butanol, isopropanol, n-propanol; sulfoxides such as dimethyl sulfoxide; amides such as dimethyl formamide, dimethyl acetamide; ketones such as acetone, methyl ethyl ketone, methyl isobutyl ketone; water and or a mixture thereof.

In one embodiment, in the step c) of the above process, (R)-ruxolitinib phosphate (the compound I-A) is obtained by a process comprising the steps of
(c-i) dissolving (R)-ruxolitinib DBTA salt, the compound XIV, in a suitable solvent to obtain a solution;
(c-ii) adding a base to the solution of step (c-i) to obtain (R)-ruxolitinib base (the compound I);
(c-iii) dissolving (R)-ruxolitinib base (the compound I) in an alcohol solvent;
(c-iv) adding a solution of phosphoric acid in an alcohol solvent to the above step (c-iii) to obtain a reaction mixture;
(c-v) stirring the reaction mixture of the above step (c-iv); and
(c-vi) isolating crystalline (R)-ruxolitinib phosphate (the compound I-A).

The alcohol solvent used in step (c-iii) may be selected from the group consisting of methanol, ethanol, ethane-1,2-diol, n-propanol, isopropanol, n-butanol, isobutanol, tert-butanol and the like.

In one embodiment, R-ruxolitinib (the compound I) obtained by treating the compound XIV with a base is treated with phosphoric acid in a suitable solvent to obtain crystalline (R)-ruxolitinib phosphate.

In one embodiment, the present invention provides a compound of formula IV (the compound IV);

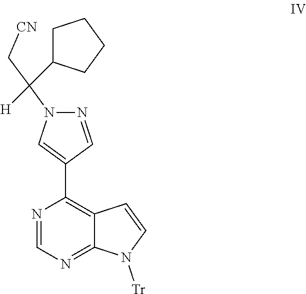

wherein “Tr” is triphenylmethyl.

In one embodiment, the present invention provides a compound IV which is characterized by ¹H NMR (CDCl₃, 400 MHz) having peaks at 8.51, 8.30, 7.30-7.26, 7.21-7.18, 6.74-6.73, 4.57-4.51, 3.1-2.98, 2.63-2.6, and 1.75-1.57.

In one embodiment, the present invention provides a compound IV which is characterized by Differential Scanning Calorimetric (DSC) thermogram having endothermic peak at about 138±3° C.

In one embodiment, the present invention provides a compound selected from the compounds of formulae X and XI (the “compound X” and the “compound XI”).

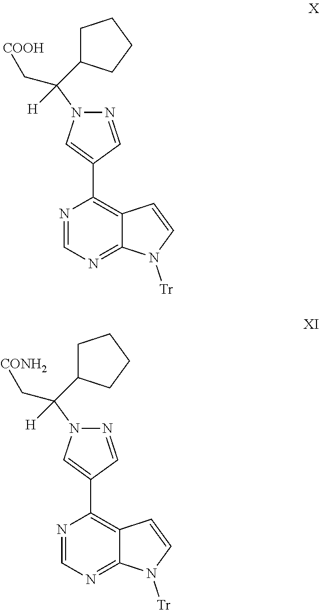

In one embodiment, the present invention provides a compound X.

In one embodiment, the present invention provides a compound XI which is characterized by ¹H NMR having peaks at 11.25, 8.86, 8.43, 8.3, 7.36-7.34, 7.14-6.98, 6.71-6.70, 4.54-4.51, 3.34-2.86, 2.46-2.24, and 1.83-1.20.

In one embodiment, the present invention provides crystalline (R)-ruxolitinib phosphate, the compound I-A, obtained by the process of the present invention having a content of compounds X, XI, XII or XIII in an amount from about 0.15%

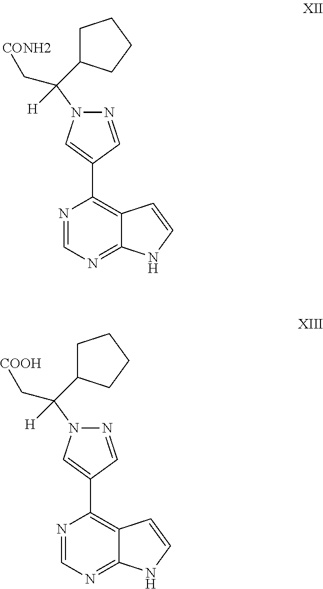 [SMILES: NC(=O)CC(C1CCCC1)n1cc(-c2ncnc3[nH]ccc23)cn1 ; O=C(O)CC(C1CCCC1)n1cc(-c2ncnc3[nH]ccc23)cn1]

to about 0.03% w/w as determined by HPLC (high performance liquid chromatography). The chemical structures of the compounds XII and XIII are represented above.

In one embodiment, the present invention provides crystalline (R)-ruxolitinib phosphate, the compound I-A having a content of compound X, XI, XII or XIII in an amount from about 0.15% to about 0.03% w/w as determined by HPLC.

In one embodiment, the present invention provides a pharmaceutical composition comprising crystalline (R)-ruxolitinib phosphate (the compound I-A) having a content of the compounds X, XI, XII or XIII in an amount from about 0.15% to about 0.03% w/w as determined by HPLC.

In one embodiment, the present invention provides a pharmaceutical composition comprising crystalline (R)-ruxolitinib phosphate having a content of compounds X, XI, XII, XIII, XVIII, XIX or XX in an amount from about 0.15% to about 0.03% w/w as determined by HPLC.

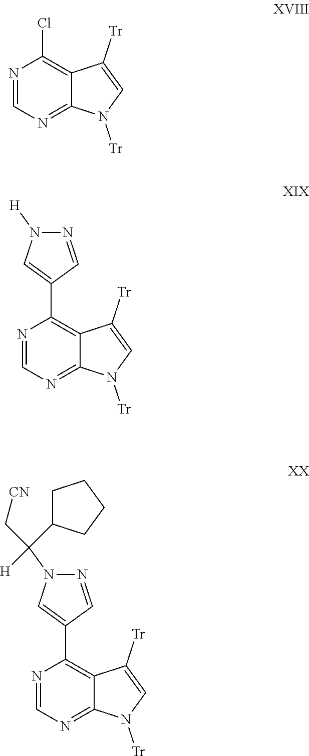

In one embodiment, the compound of formula IV (the compound IV)

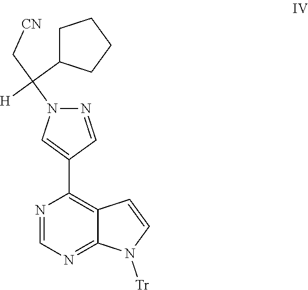

used in the step (a) of the process for the preparation of the compound I-A is obtained by a process comprising the steps of:
(1) reacting a compound of formula IX (the “compound IX”) with trityl chloride i.e. triphenylmethyl chloride in the presence of a base to obtain a compound of formula VIII (the “compound VIII”);

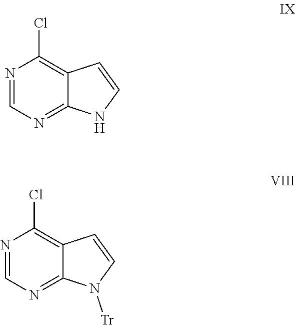

(2) reacting the compound VIII obtained in the step (1) with a compound of formula VII (the “compound VII”);

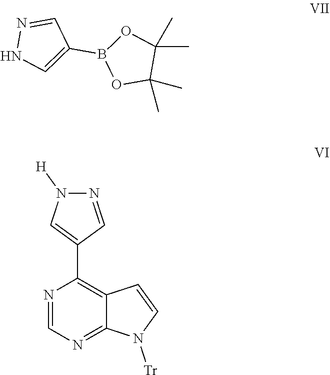

in the presence of a catalyst to obtain compound of formula VI (the “compound VI”); and (3) reacting the compound VI obtained in the step (2) with 3-cyclopentylacrylonitrile, the compound of formula V (the “compound V”)

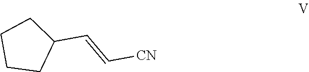

in the presence of a base to obtain the compound IV.

In one embodiment, in the step (1) of the above process, the compound IX is reacted with trityl chloride in the presence of a base and a solvent to obtain the compound VIII.

In one embodiment, the base used in step (1) may be selected from the group consisting of an organic or an inorganic base. The inorganic base may be selected from the group consisting of hydroxides such as sodium hydroxide, potassium hydroxide, calcium hydroxide, lithium hydroxide; a carbonate such as of sodium carbonate, potassium carbonate, calcium carbonate, lithium carbonate; an alkoxide such as sodium methoxide, potassium methoxide; bicarbonates such as sodium bicarbonate, potassium bicarbonate, lithium bicarbonate; ammonia and the like. The organic base may be selected from the group consisting of amines such as triethylamine, diisopropylethylamine, N, N-dimethylaniline, pyridine and the like.

In one embodiment, the solvent used in the process for the preparation of the compound IV may be selected from the group consisting of chlorinated solvents such as methylene dichloride, ethylene dichloride, chloroform, carbon tetrachloride; ethers such as diethyl ether, methyl tertiary butyl ether, di-isopropyl ether, tetrahydrofuran; nitriles such as acetonitrile, propionitrile, butyronitrile, benzonitrile; esters such as ethyl acetate, butyl acetate, isopropyl acetate; hydrocarbons such as cyclohexane, toluene, xylene, hexane; alcohols such as methanol, ethanol, butanol, isopropanol, n-propanol; sulfoxides such as dimethyl sulfoxide; amides such as dimethyl formamide, dimethyl acetamide; ketones such as acetone, methyl ethyl ketone, methyl isobutyl ketone; water and or a mixture thereof.

In one embodiment, the compound VIII obtained in the above step (1) may be optionally purified.

In one embodiment, the compound VIII may be used as such in the next process step without purifying or drying it.

In one embodiment, in step (2) of the above process, the compound VIII is reacted with a compound VII in the presence of a palladium catalyst selected from the group consisting of Tetrakis(triphenylphosphine) palladium(0) or tetrakis(tri(o-tolyl)phosphine)palladium(0), nickel (II) chloride hexahydrate with 1,3-bis (diphenylphosphino)propane in 2-propanol, [1,1′-Bis(diphenylphosphino)-ferrocene]dichloronickel(II); to obtain the compound VI.

The reaction may be carried out in the presence of an inorganic base such as sodium acetate, potassium acetate, sodium carbonate, sodim bicarbonate, potassium carbonate, sodium hydroxide, lithium hydroxide, potassium hydroxide, lithium carbonate.

The solvent used in the step (2) may be selected from those discussed supra.

In one embodiment, in the step (3) of the above process, the compound VI is reacted with the compound V in the presence of a base and a solvent to obtain the compound IV.

The base used in the step (3) may be selected from the group consisting of tetramethyl guanidine, 1,8-diazabicyclo(5.4.0)undec-7-ene, 1,5-diazabicyclo(4.3.0)non-5-ene, 1,4-diazabicyclo(2.2.2)octane, tert-butyl ammonium hydroxide, sodium hydroxide, potassium hydroxide, sodium methoxide, sodium ethoxide, tripotassium phosphate, sodium silicate, calcium oxide, triethylamine, sodium carbonate, potassium carbonate, sodium bicarbonate, potassium bicarbonate, potassium hydrogen phosphate, triphenyl phosphine, triethyl phosphine, potassium acetate, or potassium acrylate.

The solvent used in the step (3) of the above process may be selected from those discussed supra.

In one embodiment, the present invention provides a process for the racemisation of the chiral salt of the undesired enantiomer, a compound of formula XV [DBTA-(S)-enantiomer, “compound XV” ]

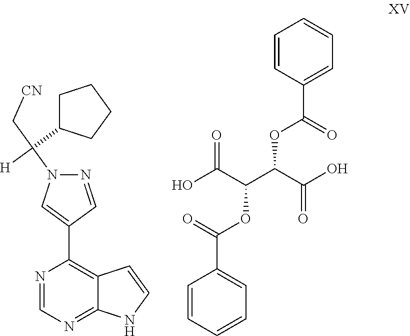

to obtain racemic mixture of ruxolitinib, the compound III, wherein the process comprises the steps of:
a-1) reacting the compound XV with a base to obtain (S)-ruxolitinib, the compound of formula XVI (the “compound XVI”);

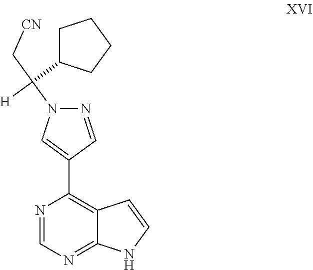

a-2) reacting the compound XVI obtained in the step (a-1) with trityl chloride in the presence of a base to obtain (S)—N-trityl ruxolitinib, the compound of formula XVII (the “compound-XVII”);

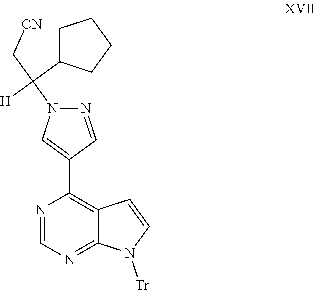

a-3) reacting the compound XVII obtained in the step (a-2) with a base in the presence of a solvent to obtain the compound VI;
a-4) reacting the compound VI obtained in the step (a-3) with the compound V in the presence of base and a solvent to obtain N-trityl ruxolitinib, the compound IV; and
a-5) reacting the compound IV obtained in the step (a-3) with an acid to obtain racemic ruxolitinib, the compound III.

The base and solvent used in the afore described process for the racemization of the compound XV are selected from those discussed supra.

The examples that follow are provided to enable one skilled in the art to practice the invention and are merely illustrative of the invention. The examples should not be read as limiting the scope of the invention.

### EXAMPLES

### General Methods:

### 1] HPLC Method (Instrumental Settings):

High performance liquid chromatography (HPLC) was performed with the conditions described below for determining purity:

### A] for Chiral Purity:

Column: Inertsil ODS 3V, 150×4.6 mm, 5μ;
Column Temperature: 30° C.,
Mobile Phase A: Adjust the pH of Water 2.50 with diluted Perchloric acid in water;
Mobile Phase B: Acetonitrile:Methanol (80:20 v/v):Buffer (900:100 v/v)
Sample Cooler temperature: 10° C.

| Time (min) | % Mobile Phase A | % Mobile Phase B |
|---|---|---|
| 0.01 | 85 | 15 |
| 45 | 05 | 95 |
| 50 | 05 | 95 |
| 51 | 85 | 15 |
| 56 | 85 | 15 |

Diluent: Acetonitrile:Methanol:Buffer (40:40:20v/v/v);
Flow Rate: 1.0 mL/Minute,
Detection wavelength: UV 225 nm;
Injection Volume: 15 μL;
Run time: 56 minutes.

### B] for Chiral Purity:

### Reagents and Solvents:

n-Hexane (Rankem Grade); 2-Propanol (Rankem Grade); Methanol (Rankem Grade); 1-Propanol (Rankem Grade).
Column: Chiralcel OD-H, 250×4.6 mm, 5μ;
Column temperature: 40° C.;
Sample cooler temperature: 10° C.;
Mobile Phase: n-Hexane:2-Propanol:Methanol:Diethylamine (780:200:20:1.0, v/v/v/v);
Diluent: n-Hexane:2-Propanol:Methanol (500:500:10, v/v/v)
Flow Rate: 0.7 mL/minute;
Detection: UV 225 nm;
Injection Volume: 20 μL:
Run time: 25 minutes.

### For Assay:

Mobile Phase A: Adjust the pH of water 2.50 with diluted Perchloric acid in water; Mobile Phase B: Acetonitrile:Methanol (80:20 v/v):Buffer (900:100 v/v)

| Time (Min) | % Mobile Phase A | % Mobile Phase B |
|---|---|---|
| 0.01 | 85 | 15 |
| 15 | 55 | 45 |
| 17 | 02 | 98 |
| 22 | 02 | 98 |
| 23 | 85 | 15 |
| 29 | 85 | 15 |

Flow rate: 1.2 mL/minute;
Detection wavelength: UV 225 nm;
Injection volume: 15 μL;
Diluent—: Acetonitrile:Methanol:Buffer (40:40:20v/v/v);
Run time: 29.0 minutes.

### 2]X-Ray Powder Diffraction (XRPD) (Instrumental Settings):

The measurements were performed on Philips X-Ray Diffractometer model XPERT-3 (PANalytical) Detector: X'celerator using Cu lamp with type and wavelength of the X-ray radiation: K-α₁ 1.54060[Å], K-α₂ 1.5444[Å] under the following conditions:

The measurements were carried out with a Pre FIX module programmable soller slit and anti-scatter Slit (Offset 0.00°); Generator settings: 40 mA/45 kV, tube current 40 mAmp Time per step: 50 s, Step size: 0.0167, Peak width 2.00 and start angle (°) 2.0 and End angle: 50.0; Scan type: continuous; measurement performed at 25° C. The XRPD instrument is calibrated using NIST SRM 640C silicon standard and NIST SRM 1976b Alumina.

Sample preparation: Take an adequate amount of the sample to fill the sample holder using back-loading technique. Then load the sample holder between the X-ray optics-path and scan using the above described parameters. Integrate the obtained powder X-ray diffraction profiles using HighScore Software.

### 3] Thermogravimetric Analysis (Instrumental Settings):

TGA thermogram was recorded using TGA-Q500 (Waters). About 5-10 mg of sample was taken in sample holder and loaded it in furnace. The sample was heated up to 250° C. at the ramp rate of 10° C./min and the thermogram was integrated by using Universal V4.5A software and calculate the weight loss by sample up to 100° C.

### Example 1: Preparation of 4-Chloro-7-(triphenylmethyl)-7H-pyrrolo [2,3-d]pyrimidine (Compound VIII)

Sodium hydroxide solution (781.7 g sodium hydroxide in 2000 mL water) was added to 4-Chloro-7H-pyrrolopyrimidine (2.0 Kg) in acetone (8.0 L) and stirred. Trityl chloride (4.359 Kg) was added in over a period of one hour to above clear solution at a temperature of 20° C.-35° C. Resulting solution was stirred for 4 to 6 hours at a temperature of 20° C.-30° C. Precipitated solid was filtered and washed with water to obtain the compound VIII.

HPLC Purity: 89.9%; Yield: 96.0%.

### Example 1a: Purification of Crude 4-Chloro-7-(triphenylmethyl)-7H-pyrrolo[2,3-d]pyrimidine (Compound VIII)

The compound VIII (10 g) from example 1 was stirred in acetone (40 mL) at reflux temperature for 30 minutes. The reaction mixture was cooled to a temperature of 20° C.-30° C. and stirred for 60 minutes. Solid obtained was filtered, washed with acetone and dried to obtain title compound with chemical purity 98.8% (Determined by HPLC).

### Example 2: Preparation of 4-(1H-Pyrazol-4-yl)-7-(triphenylmethyl)-7H-pyrrolo[2,3-d]pyrimidine (Compound VI)

To a round bottom flask water (125 mL) and 1-propanol (125 mL) were added at a temperature of 25° C.-30° C. and purged with nitrogen gas. 4-Chloro-7-triphenylmethylpyrrolo[2,3-d]-pyrimidine (Compound VIII, 50 g), 4-(4,4,5,5-tetramethyl-1,3,2-dioxaborolan-2-yl)-1H-pyrazole (Compound VII, 29.4 g), sodium acetate (20.7 g) and Tetrakis(triphenylphosphine) palladium (0) (2.9 g) were charged. The reaction mixture was heated to a temperature of 80° C.-90° C. and maintained for 4 to 8 hours. On completion of the reaction, the reaction mixture was gradually cooled to a temperature of 20° C.-30° C. The solid was filtered, washed with one volume 1:1 mixture of 1-propanol and water, dried to obtain the compound VI.

Toluene (150 mL) was added to crude compound VI and stirred at a temperature of 50° C.-55° C. for 30 minutes. Slurry was cooled to a temperature of 20° C.-30° C. and stirred for 60 minutes. Solid was filtered, washed with toluene and dried to obtain title compound with Yield: 84.4%; Chemical Purity: 95.6% (Determined by HPLC).

### Example 3: Preparation of 3-Cyclopentyl-3-{4-[7-(triphenylmethyl)-7H-pyrrolo[2,3-d]pyrimidin-4-yl]-1H-pyrazol-1-yl}propanenitrile (Compound IV)

To a round bottom flask was added 4-(1H-Pyrazol-4-yl)-7-(triphenylmethyl)-7H-pyrrolo[2,3-d]pyrimidine (Compound VI, 2000 g), dimethyl formamide (2000 mL), 3-cyclopentyl acrylonitrile (905.7 g) and 1,8-diazabicyclo[5.4.0]undec-7-ene (852.3 g) were added to reaction mixture, and heated to a temperature of 40° C.-65° C. for 6 to 10 hours. The reaction mixture was cooled to a temperature of 20° C.-30° C., followed by addition of water. The precipitated solid was filtered and washed with water and dried at a temperature of 50° C.-55° C. for 12 hours. To the obtained solid, methanol was added and stirred at 55° C.-60° C. for 30 minutes. The reaction mixture was cooled to 20° C.-30° C. The obtained solid was filtered. The wet cake was stirred with methanol at a temperature of 55° C.-60° C. for 30 minutes. The reaction mass was cooled to a temperature of 20° C.-30° C. Solid was filtered and washed with methanol. The obtained solid was dried to obtain the Compound IV.

Chemical Purity: 97.8% (Determined by HPLC); Yield 89%; Melting Point: 138° C.

### Example 3A: Telescopic synthesis of 3-Cyclopentyl-3-{4-[7-(triphenylmethyl)-7H-pyrrolo[2,3-d]pyrimidin-4-yl]-1H-pyrazol-1-yl}propanenitrile (Compound IV)

Sodium hydroxide solution (19.5 g sodium hydroxide in 50 mL water) was added to 4-chloro-7H-pyrrolopyrimidine (compound IX, 50 g) in acetone (200 mL) and stirred at a temperature of 20° C.-30° C. Trityl chloride (108.97 g) was added to the above solution at a temperature of 20° C.-35° C. The resulting solution was stirred for 4 to 6 hours at a temperature of 20° C.-30° C. The precipitated solid was filtered to obtain the compound VIII which is used as such without purification or drying in the next stage.

To a round bottom flask, water (305 mL) and 1-propanol (305 ml) were added at a temperature of 20° C.-30° C. and purged with nitrogen gas, the compound VIII obtained above, 4-(4,4,5,5-tetramethyl-1,3,2-dioxaborolan-2-yl)-1H-pyrazole (Compound VII, 71.7 g), sodium acetate (50.5 g) and Tetrakis(triphenylphosphine) palladium (0) (7.1 g) were charged. The reaction mixture was heated to a temperature of 80° C.-90° C. and maintained for 4 to 8 hours. After completion, the reaction mixture was gradually cooled to a temperature of 20° C.-30° C. to obtain a solid. The solid was filtered. To the obtained solid, toluene (366 mL) was added at a temperature of 50° C.-55° C. for 30 minutes. The obtained slurry was cooled to a temperature of 20° C.-30° C. and stirred for 60 minutes. The obtained compound VI was isolated by filtration and used as such without purification or drying for next stage.

To a round bottom flask, was added the above obtained compound VI (155 g), dimethyl formamide (112 mL), 3-cyclopentyl acrylonitrile (Compound V, 38 g) and 1,8-diazabicyclo[5.4.0]undec-7-ene (47.8 g) were added to the reaction mixture and heated to a temperature of 40° C.-65° C. for 6 to 10 hours. The reaction mixture was cooled to a temperature of 20° C.-30° C., followed by addition of water (395 mL). The precipitated solid was filtered and methanol (340 mL) was added and stirred at 55° C.-60° C. for 30 minutes. The reaction mixture was cooled to a temperature of 20° C.-30° C. The obtained solid was filtered. The wet cake was stirred with methanol at a temperature of 55° C.-60° C. for 30 minutes. The reaction mass was cooled to a temperature of 20° C.-30° C. The solid was filtered and dried to obtain the compound IV.

Chemical Purity: 97.8% (Determined by HPLC); Yield 89%.

### Example 4: Preparation of (R)-Ruxolitinib DBTA Salt (Compound XIV)

To a round bottom flask, glacial acetic acid (1000 mL) and 3-cyclopentyl-3-{4-[7-(triphenylmethyl)-7H-pyrrolo[2,3-d]pyrimidin-4-yl]-1H-pyrazol-1-yl}propanenitrile (compound IV) (500 g) and molecular sieve 3° or 4° were added (1000 g) to obtain a reaction mixture. The reaction mixture was heated to a temperature of 95° C.-105° C. and maintained for 8 to 10 hours. The reaction mixture was cooled to a temperature of 50° C.-70° C. Acetic acid was distilled under vacuum, and toluene (250 mL) was added to reaction mixture and the distillation was continued under vacuum. Toluene and methanol were added to the reaction mass and filtered through hyflo bed. To the filtrate, water and conc. HCl (1500 mL) were added, and stirred for about 10-15 min at a temperature of 25° C.-30° C. The layers were separated, and this was followed by adjusting pH to 8 to 10 using sodium hydroxide solution. The product was extracted with methylene dichloride and distilled under vacuum to obtain a residue (compound III). To the residue, acetonitrile (2500 mL) and (+) DBTA ((+)-Dibenzoyl-D-tartaric acid) (163 g) were added and then heated to a temperature of 70° C.-80° C. for 30 minutes. The reaction mixture was cooled to a temperature of 20° C.-30° C. and stirred for 120 minutes. The solid was filtered, washed with acetonitrile and dried. The solid thus obtained was purified further three times with 5% aqueous acetonitrile. Reaction mass was cooled to 20° C.-30° C. and stirred for 120 minutes. Reaction mass filtered and dried under vacuum to obtain compound XIV.

Chemical Purity ≥99.9% (Determined by HPLC), Chiral purity: R-isomer 99.89% and S isomer 0.11%.

### Example 4A:Preparation of (R)-Ruxolitinib D-DBTA Salt (Compound XIV)

To a round bottom flask, glacial acetic acid (1000 mL), 3-cyclopentyl-3-{4-[7-(triphenylmethyl)-7H-pyrrolo[2,3-d]pyrimidin-4-yl]-1H-pyrazol-1-yl}propanenitrile (compound IV, 500 g), molecular sieve 3° or 4° were added (1000 g), and triflic acid (12.5 mL) was added to the reaction mixture. The reaction mixture was heated to a temperature of 95° C.-105° C. and maintained for 8 to 10 hours. The reaction mixture was cooled to a temperature of 50° C.-70° C. Acetic acid was distilled under vacuum and ethyl acetate was charged, and the reaction mixture was filtered through hyflo bed. This was again washed with 5% aqueous sodium carbonate solution at a temperature of 20° C.-30° C. Ethyl acetate was distilled under vacuum at a temperature of 45° C.-60° C. Acetonitrile (2500 mL) and (+) DBTA (Dibenzoyl tartaric acid) (163 g) were added to the residue (compound III) and then heated to a temperature of 70° C.-80° C. for 30 minutes. The reaction mixture was cooled to a temperature of 20° C.-30° C. and stirred for 120 minutes. The solid was filtered and dried. The solid thus obtained was purified further with 5% aqueous acetonitrile. The reaction mass was cooled to a temperature of 20° C.-30° C. and stirred for 120 minutes. Reaction mass was filtered and dried under vacuum to obtain compound XIV.

Chemical Purity ≥99.9% (Determined by HPLC), Chiral purity: R-isomer 99.92% and S isomer 0.08%.

### Example 4B: Preparation of (R)-Ruxolitinib D-DBTA Salt (Compound XIV)

To a round bottom flask, glacial acetic acid (200 mL), the compound IV (100 g), n-propanol (100 ml) were added, and triflic acid (2.41 mL) was added to the reaction mixture. The reaction mixture was heated to a temperature of 95°-105° C. and maintained for 8 to 10 hours. The reaction mixture was cooled to a temperature of 50° C.-70° C. Acetic acid was distilled under vacuum and methylene dichloride was charged. This was washed with aqueous sodium carbonate solution (500 mL) at a temperature of 20° C.-30° C. The methylene dichloride layer was separated and treated with charcoal. After hyflo filtration, the layer was distilled under vacuum at a temperature of 45° C.-60° C. to obtain a residue (compound III). Acetonitrile (250 mL) and (+) DBTA ((+)-Dibenzoyl (D)-tartaric acid) (35.88 g in 250 ml acetonitrile) were added to the residue (compound III) and then heated to a temperature of 70° C.-80° C. for 30 minutes. The reaction mixture was cooled to a temperature of 20° C.-30° C. and stirred for 6 hour. The solid was filtered and dried. The solid thus obtained was dissolved in 5% aqueous acetonitrile. The reaction mass was stirred and filtered hot. The solution was then cooled to a temperature of 20° C.-30° C. and stirred for 240 minutes. The resulting reaction mass was filtered and dried under vacuum to obtain compound XIV.

Chemical purity: ≥99.9% (Determined by HPLC), Chiral purity: R-isomer 99.92% and S isomer 0.08%.

### Example 5: Preparation of (R)-Ruxolitinib Phosphate (Compound I-A)

To a round bottom flask, (R)-ruxolitinib (+)-DBTA salt (compound XIV, 20 g), methylene dichloride (MDC, 200 ml), water (200 ml) and aqueous sodium carbonate (4.15 g) were charged. The reaction mixture was stirred, followed by layer separation. The organic layer was filtered through micron filter and distilled under vacuum. To the residue, isopropyl alcohol (240 ml) was added to obtain clear solution followed by addition of Ortho phosphoric acid (3.09 g) in isopropyl alcohol (IPA, 40 ml). The reaction mixture was stirred at a temperature of 20-30° C. for 60 minutes. The solid was filtered and dried under vacuum to obtain compound (I-A) with purity ≥99.9%, Chiral purity: R-isomer 99.89% and S-isomer NMT 0.11%.

1. A process for the preparation of crystalline (R)-3-(4-(7H-pyrrolo[2,3-d]pyrimidin-4-yl)-1H-pyrazol-1-yl)-3-cyclopentylpropanenitrile phosphate, a compound of formula I-A (the compound I-A), 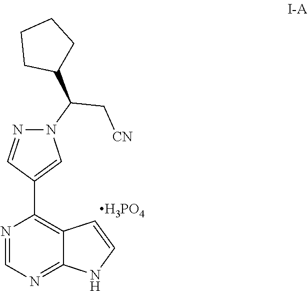
comprising the steps of:
a) reacting 3-cyclopentyl-3-{4-[7-(triphenylmethyl)-pyrrolo[2,3-d]pyrimidin-4-yl]-1H-pyrazol-1-yl}propanenitrile, a compound of formula IV (the compound IV) with an acid under anhydrous condition to obtain racemic ruxolitinib, a compound of formula III (the compound III); 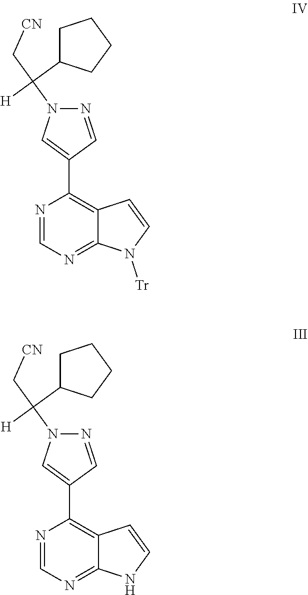
wherein Tr is triphenylmethyl;
b) reacting the compound III obtained in the step (a) with a chiral acid to obtain a chiral salt of ruxolitinib with the chiral acid, represented by a compound of formula II (the compound II); 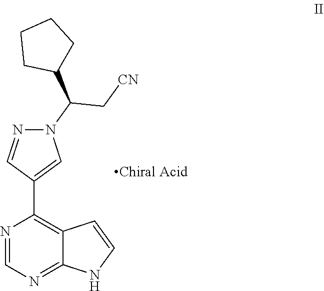 [SMILES: N#CC[C@H](C1CCCC1)n1cc(-c2ncnc3[nH]ccc23)cn1 ; "·Chiral Acid"]
c) converting the chiral salt of ruxolitinib, the compound II obtained in the step (b), to crystalline (R)-ruxolitinib phosphate, the compound I-A.

2. The process according to claim 1, wherein the chiral acid used in the step (b) is (+)-dibenzoyl-D-tartaric acid.

3. The process according to claim 1, wherein the compound IV used in the step (a) is prepared by a process comprising the steps of:
(1) reacting a compound of formula IX (the compound IX) with trityl chloride in the presence of a base to obtain a compound of formula VIII (the compound VIII); 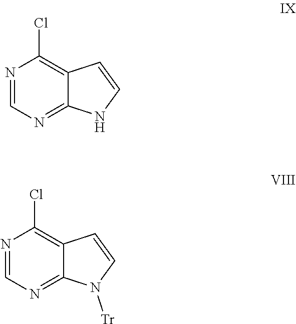
(2) reacting the compound VIII obtained in the step (1) with a compound of formula VII (the compound VII); 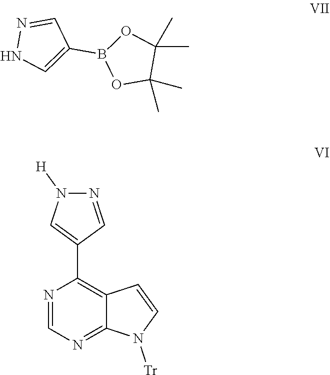
in the presence of a catalyst to obtain a compound of formula VI (the “compound VI”); and
(3) reacting the compound VI obtained in the step (2) with 3-cyclopentylacrylonitrile, a compound of formula V (the compound V) 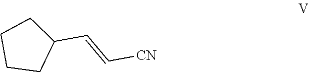
in the presence of a base to obtain the compound IV.

4. The process according to claim 3, wherein the base used in the step (1) is selected from an organic base or an inorganic base; wherein the organic base is selected from the group consisting of triethylamine, diisopropylethylamine, N, N-dimethylaniline, and pyridine; and wherein the inorganic base is selected from the group consisting of sodium hydroxide, potassium hydroxide, calcium hydroxide, lithium hydroxide, sodium carbonate, potassium carbonate, calcium carbonate, lithium carbonate, sodium methoxide, potassium methoxide, sodium bicarbonate, potassium bicarbonate, and lithium bicarbonate.

5. The process according to claim 3, wherein the catalyst used in the step (2) is a palladium catalyst selected from the group consisting of Tetrakis(triphenylphosphine) palladium(0), tetrakis(tri(o-tolyl)-phosphine)palladium(0), nickel (II) chloride hexahydrate with 1,3-bis (diphenylphosphino)propane in 2-propanol, and [1,1′-Bis(diphenyl-phosphino)ferrocene]dichloronickel(II).

6. The process according to claim 1, wherein the step c) involving the converting of the chiral salt of ruxolitinib, the compound II, to crystalline (R)-ruxolitinib phosphate, the compound I-A, comprises the steps of:
(c-i) dissolving the chiral salt of ruxolitinib, in a solvent to obtain a solution;
(c-ii) adding a base to the solution of step (c-i) to obtain a (R)-ruxolitinib base (the compound I);
(c-iii) dissolving the (R)-ruxolitinib base (the compound I) in a first alcohol solvent;
(c-iv) adding a solution of phosphoric acid in a second alcohol solvent to the step (c-iii) to obtain a reaction mixture;
(c-v) stirring the reaction mixture of the step (c-iv); and
(c-vi) isolating crystalline (R)-ruxolitinib phosphate (the compound I-A).

7. A compound selected from:
a compound of formula IV (the compound IV), 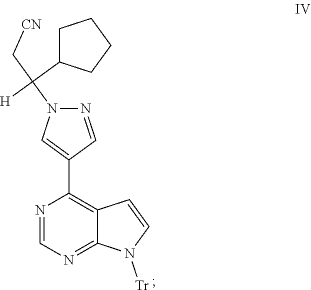
a compound of formula X, 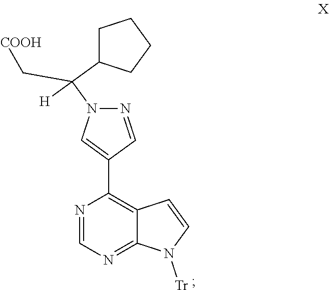
or a compound of formula XI, 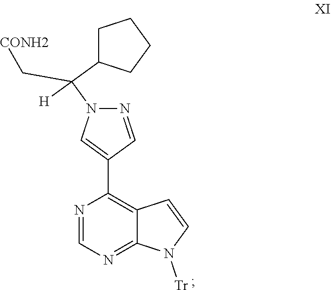
wherein Tr is triphenylmethyl.

8. (canceled)

9. The process according to claim 1, wherein the crystalline (R)-ruxolitinib phosphate, the compound I-A, obtained in the step (c) has a content of one of the compounds X, XI, XII or XIII represented by the following chemical structures, 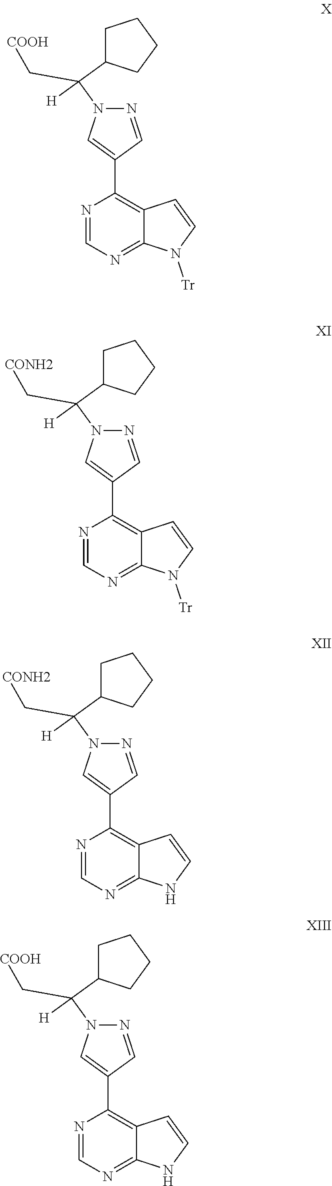
wherein in the compounds of formulae X and XI, Tr is triphenylmethyl,
in an amount from about 0.15% to about 0.03% w/w as determined by HPLC (High Performance Liquid Chromatography).

10. Crystalline R-ruxolitinib phosphate, the compound I-A, having a content of one of the compounds X, XI, XII or XIII as defined in claim 9, in an amount from about 0.15% to about 0.03% w/w as determined by HPLC.

11. The process according to claim 6, wherein the chiral salt of Ruxolitinib of step (c-i) is (R)-ruxolitinib (+)-dibenzoyl-D-tartaric acid (DBTA) salt, the compound XIV.
